(19) United States
(12) Patent Application Publication
(10) Pub. No.: US 2012/0029202 A1
(43) Pub. Date: Feb. 2, 2012

(54) Process for the Preparation of 5-Substituted 3-Aryl-3-(trifluoromethyl)-3,4-dihydro-2H-pyrroles

(75) Inventors: Koichi ARAKI, Ibaraki (JP); Jun Mihara, Oyama-shi (JP); Norio Sasaki, Inashiki-gun (JP); Peter Bruechner, Oyama-shi (JP); Kei Domon, Oyama-shi (JP); Johannes-Rudolf Jansen, Monheim (DE); Norbert Lui, Odenthal (DE)

(73) Assignee: Bayer CropScience AG, Monheim (DE)

(21) Appl. No.: 13/105,613

(22) Filed: May 11, 2011

Related U.S. Application Data
(60) Provisional application No. 61/333,528, filed on May 11, 2010.

(30) Foreign Application Priority Data
May 11, 2010 (EP) 10162515.0

(51) Int. Cl. C07D 207/20; C07D 403/10

(52) U.S. Cl. 548/265.8; 548/565

(57) ABSTRACT

The present invention relates to a process for the manufacturing of 5-substituted 3-aryl-3-(trifluoromethyl)-3,4-dihydro-2H-pyrroles of formula (I)

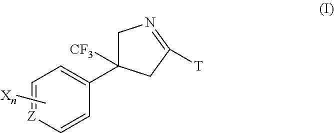

which comprises the following steps:
(i) reacting a compound of formula (II) with a isonitrile compound of formula (III)

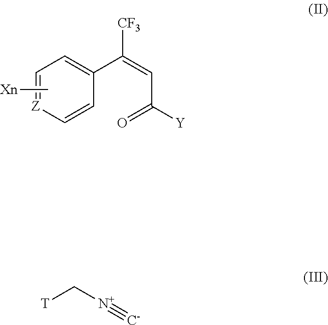

in the presence of a metal catalyst to obtain compounds of formula (IV)

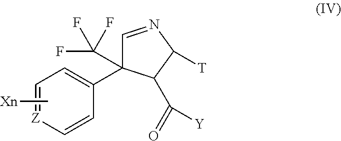

(ii) isomerizing compounds of formula (IV) to obtain compounds of formula (V) and

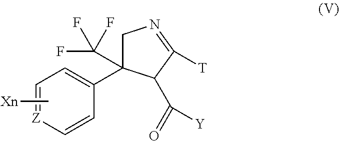

(iii) reacting the dihydropyrrole compound of formula (V) under acidic conditions, to obtain the compounds of formula (I), wherein X, n, Z, Y, and T are as defined in the description.

The present invention relates to a process for the manufacturing of 5-substituted 3-aryl-3-(trifluoromethyl)-3,4-dihydro-2H-pyrroles and two intermediates used in this process.

5-Substituted 3-aryl-3-(trifluoromethyl)-3,4-dihydro-2H-pyrroles are useful building blocks (intermediates) for the preparation of insecticidal active compounds (cf. WO2009/072621, WO2009/097992, WO2010/020522). WO2009/97992 describes the preparation of 5-substituted 3-aryl-3-(trifluoromethyl)-3,4-dihydro-2H-pyrroles starting from a styrole derivative (cf. reaction scheme 1).

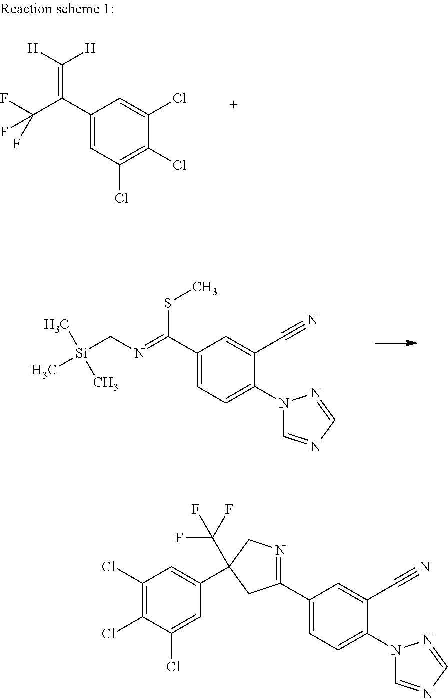

The respective styrole derivative is, however, not easily accessible and its synthesis involves many steps. Additionally, the preparation method described in WO2009/97992 does not result in chiral products but only in mixtures of different optical isomers, so-called racemic mixtures. It is, however, generally known that sometimes only specific optical or geometrical isomers of a racemic mixture show the desired biological activity. Thus, there is a need to obtain specific isomers (enantiomer) in a sufficient amount and/or in an economically advantageous way.

One way of obtaining such specific isomers is the separation of racemic compounds through chiral column chromatography. Another way is to find a preparation method through which the desired isomer can be prepared and obtained in a favorable yield.

Thus, the object of the present invention is to provide an economically advantageous new preparation method for 5-substituted 3-aryl-3-(trifluoromethyl)-3,4-dihydro-2H-pyrrole compounds.

The present inventors now found a new and economically advantageous preparation method for the preparation of 5-substituted 3-aryl-3-(trifluoromethyl)-3,4-dihydro-2H-pyrroles.

Thus, the invention is directed to a preparation method for the preparation of 5-substituted 3-aryl-3-(trifluoromethyl)-3,4-dihydro-2H-pyrroles of formula (I)

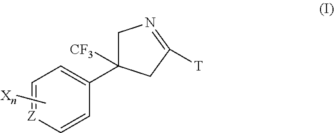

wherein T stands for one of the following chemical grouping (T-1), (T-2), (T-3)

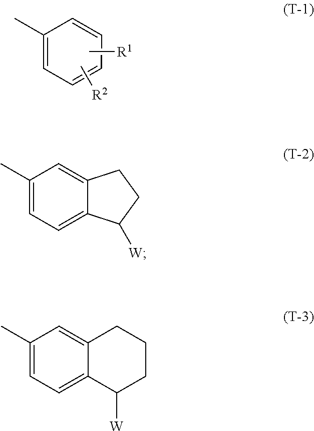

n stands for 1, 2, 3, 4, or 5,
Z stands for a carbon or a nitrogen atom which may be substituted with a substituent X,
X stands for halogen, C₁-C₁₂-alkyl, C₁-C₁₂-haloalkyl or C₁-C₁₂-haloalkoxy
W stands for —NHCOR⁶, ═NOR⁷, ═O or —OCOR⁸,
R¹ and R² independently of each other stand for hydrogen, halogen, C₁-C₁₂-alkyl, C₁-C₁₂-haloalkyl, C₁-C₁₂-alkoxy, C₁-C₁₂-alkoxycarbonyl, CH₂NHCOR³, CH(CH₃)NHCOR³, CONHR⁴, CH₂OCOR⁵, CH(CH₃)OCOR⁵, cyano, nitro, triazolyl or tetrazolyl,
R³, R⁵, R⁶, R⁷ and R⁸, independently of each other, stand for C₁-C₁₂-alkyl, C₂-C₁₂-alkenyl, arylalkyl, C₁-C₁₂-haloalkyl, C₃-C₇-cycloalkyl, C₃-C₇-cycloalkyl(C₁-C₁₂)-alkyl, C₁-C₁₂-alkyl(C₁-C₁₂)-thioalkyl, C₁-C₁₂-alkylsulfinyl(C₁-C₁₂)-alkyl, C₁-C₁₂-alkylsulfonyl(C₁-C₁₂)-alkyl, optionally substituted phenyl or optionally substituted 3- to 7-membered heterocycle containing at least one nitrogen, oxygen or sulfur atom, and
R⁴ stands for C₁-C₁₂-alkyl, C₁-C₁₂-alkenyl, arylalkyl, C₁-C₁₂-haloalkyl, C₃-C₇-cycloalkyl, C₃-C₇-cycloalkyl(C₁-C₁₂)-alkyl or a 3- to 7-membered heterocycle containing at least one nitrogen, oxygen or sulfur atom,
   which preparation method comprises the steps of
   (i) reacting a chiral trifluoromethyl-substituted acrylic compound having the general formula (II)

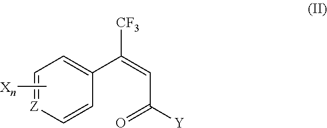

wherein
X, n, and Z have the meanings as defined above, and
Y stands for a group —OR⁹ or —NR¹⁰R¹¹, in which
R⁹ and R¹¹ independently of each other stand for an optically active C₃-C₁₂-cycloalkyl group, or an optically active —CR¹²R¹³R¹⁴ group,
R¹⁰ stand for hydrogen or C₁-C₁₂-alkyl, and
R¹², R¹³ and R¹⁴ independently from each other stand for hydrogen, C₁-C₁₂-alkyl, C₁-C₁₂-alkoxycarbonyl, C₃-C₇-cycloalkyl or an optionally substituted phenyl,
   with an isonitrile compound of the general formula (III)

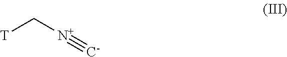

wherein T has the meaning as defined above,
in the presence of a metal catalyst, and optionally in the presence of a base, to obtain a dihydropyrrole compound having the general formula (IV)

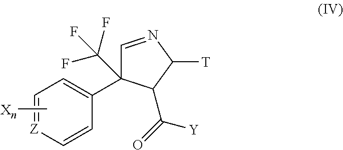

wherein X, n, Z, Y, and T have the meanings as defined above;
(ii) isomerizing a compound of formula (IV) to obtain a dihydropyrrole compound having the general formula (V)

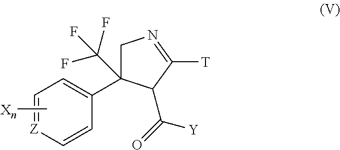

wherein X, n, Z, Y and T have the meanings as defined above;
and
(iii) reacting the dihydropyrrole compound of formula (V) under acidic conditions, to obtain the compound of formula (I).

The preparation method according to the invention is illustrated by the following reaction scheme:

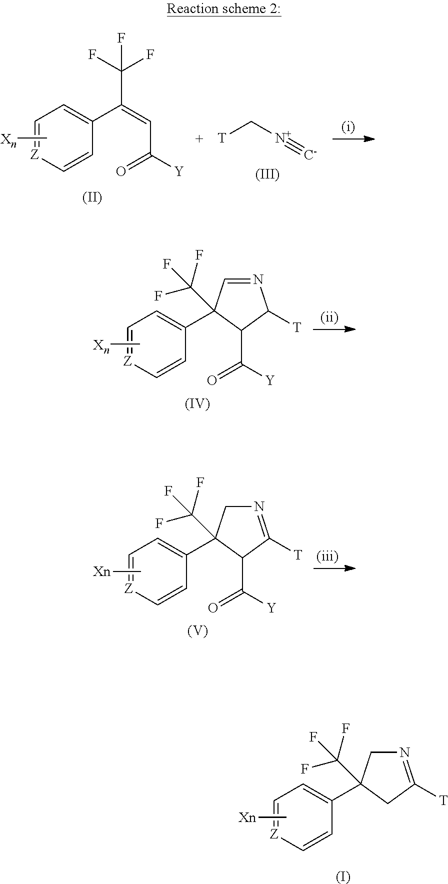

Compounds of formula (I), wherein T stands for T-1 are preferably prepared by the method according to the invention.

If not mentioned otherwise, the term “alkyl” refers to linear or branched hydrocarbon groups having from 1 to 12 carbon atoms. Examples are methyl, ethyl, n- or isopropyl, n-, iso-, sec- or tert-butyl, n-pentyl, n-hexyl, n-heptyl, n-octyl, n-nonyl, n-decyl, n-undecyl, and n-dodecyl. Preference is given to C₁-C₆-alkyl; particular preference is given to C₁-C₄-alkyl.

If not mentioned otherwise, the term “alkenyl” is defined as linear or branched hydrocarbon groups having from 2 to 12 carbon atoms and which contain at least one double bond. Examples are vinyl, allyl, 1-propenyl, isopropenyl, 1-butenyl, 2-butenyl, 3-butenyl, 1,3-butadienyl, 1-pentenyl, 2-pentenyl, 3-pentenyl, 4-pentenyl, 1,3-pentadienyl, 1-hexenyl, 2-hexenyl, 3-hexenyl, 4-hexenyl, 5-hexenyl, and 1,4-hexadienyl. Preference is given to C₂-C₆-alkenyl; particular preference is given to C₂-C₆-alkenyl.

If not mentioned otherwise, the term “alkynyl” is defined as linear or branched hydrocarbon groups having from 2 to 12 carbon atoms and which contain at least one triple bond and optionally additionally one or more double bonds. Examples are ethynyl, 1-propynyl, and propargyl. Preference is given to C₂-C₆-alkynyl; particular preference is given to C₂-C₆-alkynyl.

Each alkyl constituent in the “alkoxy”, “alkoxyalkyl”, “haloalkyl”, “cycloalkylalkyl”, “halocycloalkylalkyl”, “arylalkyl” groups and similar groups is as defined herein for “alkyl”. The same applies to groups containing an alkenyl or alkynyl, constituent.

If not mentioned otherwise, the term “cycloalkyl” is defined as cyclic hydrocarbon groups having from 3 to 12 carbon atoms. Examples are cyclopropyl, cyclobutyl, cyclopentyl, cyclohexyl, cycloheptyl, and cyclooctyl. Preference is given to C₃-C₈-cycloalkyl; particular preference is given to C₃-C₆-cycloalkyl. The term “cycloalkyl” includes such cycloalkyl groups which are optically active (“optical active cycloalkyl groups”). In view of optical active cycloalkyl groups, preference is given to optically active C₁-C₁₂-cycloalkyl groups. Examples are menthyl or bornyl. Preference is given to menthyl. Such cycloalkyl groups may be substituted by suitable substituents. Such substituents include C₁-C₁₂-alkyl groups. Examples of such optically active substituted C₁-C₁₂-cycloalkyl groups are (1R,2S,5R)-2-isopropyl-5-methylcyclohexyl or (1S,2R,5S)-2-isopropyl-5-methylcyclohexyl.

If not mentioned otherwise, the term “heterocycle” used either alone or combined with other terms stands for a 3-, 4-, 5-, 6- or 7-membered heterocyclic group containing at least one of N, O and S as a heteroatom. Typically a heterocyclic group contains no more than 4 nitrogen atoms, no more than 2 oxygen atoms and/or no more than 2 sulfur atoms. The cyclic group or the ring can be saturated, unsaturated or partially saturated. If not mentioned otherwise, a heterocyclic group can be attached to a main part through any available carbon or heteroatom of the heterocyclic group. The term additionally includes fused heterocyclic group which may be benzo-condensed. The heterocyclic group includes, for example, oxiranyl, thiiranyl, aziridinyl, oxetanyl, thietanyl, azetidinyl, furyl, thienyl, pyrrolyl, isoxazolyl, pyrazolyl, oxazolyl, oxathiazolyl, imidazolyl, triazolyl, oxadiazolyl, thiadiazolyl, tetrazolyl, pyridyl, pyrimidinyl, pyridazinyl, pyrazinyl, triazinyl, indolyl, benzoxazolyl and quinolyl.

If not mentioned otherwise, the term “aryl” refers to aromatic rings having from 6 to 14 carbon atoms. Preference is given to phenyl.

If not mentioned otherwise, the term “arylalkyl” or “aralkyl” refers to alkyl-containing aryl groups. Examples are benzyl, tolyl, xylyl, phenylethyl or α-methylbenzyl. Preference is given to benzyl and as optically active arylgroup to α-methylbenzyl.

The term “halogen” refers to fluorine, chlorine, bromine or iodine. Preference is given to fluorine or chlorine.

The term “halogen” or “halo” used either alone or contained in other terms such as “haloalkyl” includes fluorine, chlorine, bromine or iodine.

If not mentioned otherwise, the term “haloalkyl” used either alone or combined with other terms refers to alkyl groups which are partially or fully substituted with halogen atoms which may be the same or different. Examples of “haloalkyl” include CF₃, CH₂F, CHF₂, CH₂CHF₂, CCl₃, CH₂Cl, CHCl₂, CF₂CF₃, CH₂CF₃, CH₂CH₂Cl, CH₂CH₂F, CHClCH₃, CHFCH₃, CH₂CHFCl, CHCl₂, CF₂CF₂H, CH₂CF₃.

Among the trifluoromethyl-substituted acrylic compounds of formula (II) used in step (i)

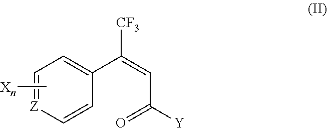

compounds are preferred,
wherein
X independently of each other stands for halogen, C₁-C₁₂-alkyl, C₁-C₁₂-haloalkyl or C₁-C₁₂-haloalkoxy, in particular for C₁₋C₆-alkyl, F, Cl, Br, I, trifluoromethyl, difluoromethoxy, methoxy, more particular for trifluoromethyl, methyl, difluoromethoxy, F, Cl, or Br; and
n stands for 1, 2, 3, 4, or 5;
Z stands for a carbon or a nitrogen atom which may be substituted with a substituent X, in particular for an optionally substituted carbon atom; and
Y stands for OR⁹ or NR¹⁰R¹¹, wherein
R⁹ and R¹¹ independently of each other, stand for an optically active C₃-C₁₂-cycloalkyl group, or an optically active —CR¹²R¹³R¹⁴ group, in particular for menthyl or α-methylbenzyl,
R¹⁰ stand for hydrogen or C₁-C₁₂-alkyl.

Compounds of formula (II) can be prepared by using trifluoromethyl-substituted acrylic acids having the general formula (VI)

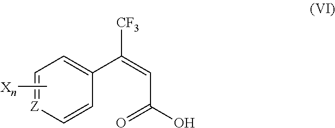

wherein X, n, and Z are as defined herein, and which are commercially available or can be synthesized by methods known in the art (Tetrahedron Letters 44 (2003) 7119-7120; Journal of Fluorine Chemistry 127 (2006) 850-853).

The compounds of formula (VI) are reacted under standard conditions to the corresponding acid chloride by using a chlorination agent, such as thionylchlorid or oxalyl chloride. The resulting acid chloride compound is then reacted to the corresponding trifluoromethyl-substituted acrylic compound of formula (II) through the reaction with a chiral alcohol, such as menthol or a Chiral amine, such as 1-phenethylamine.

The isonitriles having the general formula (III) are commercially available or can be prepared by methods known in the art (Tetrahedron Letters 29 (27), (1988) 3343-3346; Heterocycles 31 (1990), 1855-1860; Journal of Organic Chemistry 70 (2005) 3542-3553; Organic & Biomolecular Chemistry, 1 (9), (2003), 1475-1479).

The reaction of steps (i), (ii) and (iii) can be conducted at reduced pressure (below 1 bar), under vacuum (below 0.4 bar), under increased pressure (above 1 bar) or under normal pressure (i.e. around 1 bar).

The reaction of step (i) can be conducted in the absence or in the presence of a solvent. It is preferred that the reaction of step (i) is conducted in the presence of a solvent. Suitable solvents are known in the art and comprise for example aliphatic and aromatic hydrocarbons (e.g. n-hexane, benzene, toluene, xylene) which can be substituted by fluorine or chlorine (e.g. methylenchloride, dichlormethane, fluorobenzene, chlorobenzene or dichlorobenzene); ether (e.g. diethylether, diphenylether, methyl-tert-butylether, isopropylethylether, dioxane, dimethylglycol or THF); nitriles (e.g. methylnitile, butylnitrile or phenylnitrile); and alcohols (e.g. ethanol or isopropanol). Preferred solvents are benzene, toluene and xylene.

The reaction of step (i) conducted in the presence of a metal catalyst and optionally in the presence of base. Examples of such metal catalysts include copper(I)oxid, copper(I)cyanide, copper(I)chloride, copper(I)bromide, copper(I)iodide, copper(I)acetate, copper(II)cyanide; copper(II)chloride, copper(II)bromide, copper(II)iodide, copper(II)oxide, copper(II)acetate, copper(II)acetylacetonate. Preferred catalyst are copper(I)oxide, copper(II)oxide, copper(II)acetylacetonate, copper(II)acetate, copper(I)cyanide. Preferred catalysts are copper(I)oxide, copper(II)oxide, copper(II)acetylacetonate, copper(II)acetate.

Examples of the bases which may be used in step (i) include alkali metal bases (e.g. lithium hydride, sodium hydride, potassium hydride, butyl lithium, tert-butyl lithium, trimethylsilyl lithium, lithium hexamethyldisilazide, sodium carbonate, potassium carbonate, cesium carbonate, tripotassium phosphate, sodium acetate, potassium acetate, sodium methoxide, sodium ethoxide, sodium-tert-butoxide and potassium-tert-butoxide), organic bases (e.g. triethylamine, diisopropylethylamine, tributylamine, N-methylmorpholine, N,N-dimethylaniline, N,N-diethylaniline, 4-tert-butyl-N,N-dimethylaniline, pyridine, picoline, lutidine, diazabicyclooctan (DABCO), diazabicyclononen (DBN), diazabicycloundecen (DBU) and imidazole)

The reaction of step (i) can be conducted at a temperature in the range from about 20° C. to about 200° C. Preferably, the reaction is conducted at a temperature in the range from about 40° C. to about 150° C., particularly from about 50° C. to about 130° C. at normal pressure. The reaction time for said reaction is between 0.5 and 20 hours. Extending the reaction time is also possible. If necessary, the solvent used in reaction step (i) is removed by distillation at normal pressure, or at a reduced pressure and at a temperature in the range from about 20° C. to about 35° C.

The reaction of step (ii), i.e. the isomerisation of compounds of formula (IV) can be conducted in an inert solvent. Suitable solvents include for example aliphatic and aromatic hydrocarbons (e.g. N-hexan, benzene, toluene, or xylene), which may be substituted by fluorine or chlorine atoms (e.g. methylenechloride, dichlormethane, trichlormethane, CCl₄, fluorbenzene, chlorobenzene or dichlorobenzene); ether (e.g. diphenylether, methyl-tert-butylether, isopropylethylether, dioxane, dimethylglycol or THF); nitriles (e.g. acetonitrile, butylnitrile or phenylnitrile); amines (e.g. pyridine, picoline, lutidine, collidine or triethyl amine); amides (e.g. Dimethyl formamide or Dimethyl acetoamide) or mixtures thereof. Among these solvents, THF, acetonitrile, pyridine and toluene are preferred.

The reaction of step (ii) is preferably conducted in the presence of a catalyst. Preferred catalysts are bases. Suitable bases are organic and inorganic bases which are usually used in such a reaction. It is preferred to use alcoholates, acetates, fluorides, phosphates, carbonates and hydrogencarbonates of alkaline or earthalkaline metals (e.g lithium hydride, sodium hydride, potassium hydride, butyl lithium, tert-butyl lithium, trimethylsilyl lithium, lithium hexamethyldisilazide, sodium carbonate, potassium carbonate, cesium carbonate, tripotassium phosphate, sodium acetate, potassium acetate, sodium methoxide, sodium ethoxide, sodium-tert-butoxide and, potassium-tert-butoxide), or tertiary amines (e.g. trimethylamin, triethylamin, tributylamin, N,N-dimethylanilin, N,N-dimethylbenzylamin, pyridine, alkylpyridine, such as 2-methyl-5-ethylpyridin, N-methylpiperidin, N-methylpyrolidon, N,N-dimethylaminopyridin, diazabicyclooctan (DABCO), diazabicyclononen (DBN) and diazabicycloundecen (DBU)). Particularly preferred are sodium methanolate, potassium-tert-butanolate, potassium carbonate and 1,8-diazabicyclo(5,4,0)undec-7-ene.

When a base is used as catalyst in reaction step (ii), then the molar ratio of the base to the resulting dihydropyrrole compound of formula (IV) is in the range from 0.05 to 10, preferably from 0.1 to 6, more preferably from 0.1 to 2. Using a higher amount of base is in principle possible, however, economically disadvantageous.

The reaction of step (ii) can be conducted at temperatures in the range of about −20° C. to about 200° C., preferably in the range of about 5° C. to about 100° C. Although longer reaction times are possible, the reaction time of step (ii) is normally in the range of about 1 hour to about 30 hours.

Compounds of formula (V) can be isolated by crystallization or through chromatography.

The reaction of step (iii), i.e. the reaction of compounds of formula (V) to obtain compounds according to the invention of formula (I) can be conducted in the presence of solvents (diluents). Solvents are preferably used in such an amount, that the reaction mixture can be stirred during the whole preparation. Preferably solvents are used which are organic solvents and which are inert under the reaction conditions.

Examples of such solvents include halogenated hydrocarbons (e.g. chlorated hydrocarbons such as tetrachlorethylene, tetrachlorethane, dichlorpropane, methylenchlorid, dichlorbutane, chloroform, CCl₄, trichlorethane, trichlorethylene, pentachlorethane, difluorbenzene, 1,2-dichlorethane, chlorbenzene, brombenzene, dichlorbenzene chlortoluene, trichlorbenzene); ether (e.g. ethylpropylether, methyl-tert-butylether, n-butylether, anisol, phenetol, cyclohexylmethylether, dimethylether, diethylether, dimethylglycol diphenylether, diproplether, diisopropylether, di-n-butylether, diisobutylether, diisoamylether, ethylene glycol dimethylether, isopropylethylether, methyl-tert-butylether, tetrahydrofuran, methyl-tetrahydrofuran, dioxan, dichlordiethylether and polyether of ethylenoxide and/or of propyleneoxid; hydrocarbons containing a NO₂ group (e.g. nitromethane, nitroethane, nitropropane, nitrobenzene, chloronitrobenzene, o-nitrotoluene); nitrites (e.g. acetonitrile, propionitrile, butyronitrile, isobutyronitrile, benzonitrile, phenylnitrile, m-chlorobenzonitrile); tetrahydrothiophendioxide and dimethylsulfoxide, tetramethylensulfoxide, dipropylsulfoxide, benzylmethylsulfoxide, diisobutylsulfoxide, dibutylsulfoxide, diisoamylsulfoxide; sulfones (e.g. dimethyl-, diethyl-, dipropyl-, dibutyl-, diphenyl-, dihexyl-, methylethyl-, ethylpropyl-, ethylisobutyl- and pentamethylensulfone); aliphatic, cyclic aliphatic or aromatic hydrocarbons (e.g. pentan, n-hexan, n-heptan, n-oktan, nonan, “white spirits” having components with a boiling point in the range of about 40° C. to 250° C., cymol, fraction of benzine which are boiling in an interval from 70° C. to 190° C., cyclohexane, methylcyclohexane, petrolether, ligroin, octan, benzene, toluene, xylene; acides (e.g. formic acid, acetic acid, propionic acid); ester (e.g. methyl-, ethyl-, butyl-, isobutylacetate, dimethyl-, dibutyl-, ethylencarbonate); amides (e.g. hexamethylene phosphoric acid triamide, formamide, N,N-dimethyl-acetamide, N-methyl-formamide, N,N-dimethyl-formamide, N,N-dipropyl-formamide, N,N-dibutyl-formamide, N-methyl-pyrrolidine, N-methyl-caprolactame, 1,3-dimethyl-3,4,5,6-tetrahydro-2(1h)-pyrimidine, octylpyrrolidone, octylcaprolactame, 1,3-dimethyl-2-imidazolindion, n-formyl-piperidine, n,n′-1,4-diformyl-piperazin); and aliphatic alcohols (e.g. methanol, ethanol, n-propanol, iso-propanol and n-butanol).

The reaction step (iii) is preferably conducted in a solvent which is selected among the following solvents: dioxan, butyronitril, propionitril, acetonitril, ethylene glycol dimethylether, toluene, xylene, THF, dichlorbenzene, chlorbenzene, n-heptane, iso-butanol, n-butanol, ethanol, methyl-tert-butylether, isopropylethylether, acetic acid and mixtures thereof.

Depending on the starting materials used, it is also possible that the reaction of step (iii) is conducted without solvents.

The reaction of step (iii) is conducted under acidic conditions, which means that compounds of formula (V) are reacted at a pH lower than 7. Acidic conditions can be achieved by the addition of an acid, preferably a Brønstedt acid. Among the Brønstedt acids, organic and inorganic acids may be used. Suitable organic acids are trifluoroacetic acid, acetic acid, methanesulfonic acid and p-toluenesulfonic acid. It is preferred to use inorganic acids such as H₃PO₄, H₂SO₄, HCl, HBr, HF or KHSO₄. The acids can be used in a concentrated form (e.g. no water is present) or in a diluted form (e.g. as 85% H₃PO₄ or 37% HCl). It is preferred to use the acids in the concentration which is commercially available.

The reaction temperature in the reaction of step (iii) may vary. In general, the reaction of step (iii) can be carried out at temperatures in the range of about 20° C. to about 200° C., preferably in the range of about 20° C. to about 150° C.

At the end of the reaction of step (iii), it is favorable to remove the water which is synthesized during the reaction, through distillation of an azeotropic mixture. If solvents are used having a high boiling point, then such a distillation can be conducted in vacuum. By doing so, a quantitative reaction is generally achieved.

In case the reaction is conducted in a solvent, then the solvent is removed from the reaction mixture by distillation after the reaction is finished. Removal of the solvent can be done at normal pressure or reduced pressure at room temperature or increased temperature. Compounds of general formula (I) can be isolated through crystallization.

The preparation method according to the invention and preparation methods for selected starting materials are illustrated through the following examples without restricting the present application to these examples.

### EXAMPLE 1

### Preparation of (2E)-4,4,4-trifluoro-3-(3,4,5-trichlorophenyl)but-2-enoic acid to be used as starting compound for the preparation as described in following examples

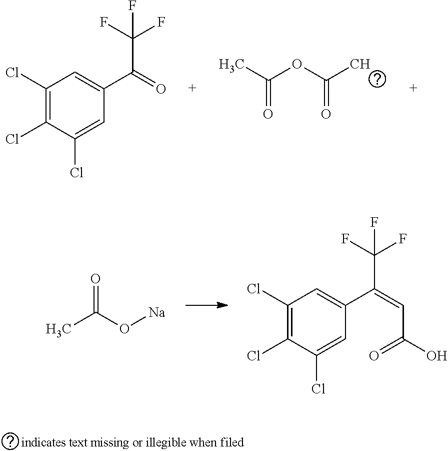

A mixture of 2,2,2-trifluoro-1-(3,4,5-trichlorophenyl)ethanone (20 g, 72 mmol), acetic anhydride (100 ml) and sodium acetate (11.8 g, 144 mmol) was stirred at 100° C. for 5 hours. The reaction mixture was cooled to 4° C. and water (200 ml) was added over 30 minutes. The mixture was warmed up to room temperature, stirred for 2 hours and stood over night. After that the mixture was extracted with methyl-tert-butylether (150 ml×3). The combined organic fractions were washed with brine, dried over anhydrous magnesium sulfate, concentrated at reduced pressure and residual AcOH was removed with toluene. 30 g of crude product was obtained.

The crude product was purified via re-crystallization (using a hexane-toluene mixture). 17.9 g of title compound was obtained as a white solid (85.5% yield).

The mother liquid was evaporated and re-crystallized again to obtain another 2.6 g of title product as a white solid (11.3% yield).

Both of them were single isomer as judged by proton NMR.

¹H-NMR (CDCl₃+DMSO-d⁶) δ: 6.67 (1H, s), 7.35 (2H, s)

### EXAMPLE 2

### Preparation of (1R,2S,5R)-2-isopropyl-5-methylcyclohexyl (2E)-4,4,4-trifluoro-3-(3,4,5-trichlorophenyl)but-2-enoate

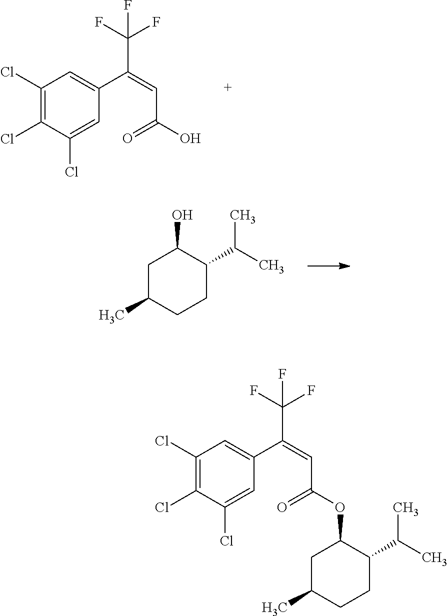

### (1) Preparation of the Acid Chloride

A mixture of (2E)-4,4,4-trifluoro-3-(3,4,5-trichlorophenyl)but-2-enoic acid (5.0 g, 14 mmol), dichloromethane (50 ml) and a catalytic amount of N,N-dimethylformamide was stirred at room temperature (around 20° C.), a solution of oxalyl chloride (2.7 g, 21 mmol) in dichloromethane (20 ml) was added drop wise.

The reaction was allowed to stir at the same temperature for 1 hour. The solvent was removed under reduced pressure. Residual solvent was removed with toluene to obtain the corresponding acid chloride, which was used for the next step without further purification.

### (2) Preparation of the Title Compound of Formula (II)

Dichloromethane (50 ml) and (1R,2S,5R)-2-isopropyl-5-methylcyclohexanol (2.64 g, 17 mmol) were added to the not purified (crude) acid chloride then stirred on an ice-water bath. Triethylamine (2.14 g, 21 mmol) was added drop wise. The reaction was allowed to warm up to room temperature and then stirred for 1 hour.

For working up the reaction mixture, water (100 ml) was added and the phases were separated. The water phase was extracted with dichloromethane (50 ml×2). The combined organic phases were washed with brine then dried over MgSO₄. Solvent was removed under reduced pressure. The crude product was then purified with silica gel column chromatography (hexane/ethyl acetate—98/2) to obtain title compound (6.07 g, 94% yield).

¹H-NMR (CDCl₃) δ: 0.65-1.87 (18H, m), 4.59-4.68 (1H, m), 6.66 (1H, s), 7.32 (2H, s).

### EXAMPLE 3

### Preparation of (1R,2S,5R)-2-isopropyl-5-methylcyclohexyl 2-(3-bromo-4-fluorophenyl)-4-(3,4,5-trichlorophenyl)-4-(trifluoromethyl)-3,4-dihydro-2H-pyrrole-3-carboxylate

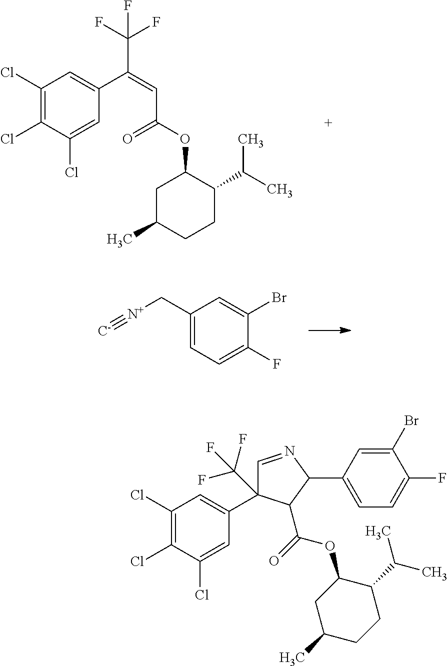

A mixture of (1R,2S,5R)-2-isopropyl-5-methylcyclohexyl (2E)-4,4,4-trifluoro-3-(3,4,5-trichlorophenyl)but-2-enoate (800 mg, 1.75 mmol), 2-bromo-1-fluoro-4-(isocyanomethyl)benzene (380 mg, 1.75 mmol) copper(I)oxide (12.5 mg, 0.09 mmol) and toluene (20 ml) was refluxed for 11 hours. The reaction mixture was poured into water (50 ml) then extracted with ethyl acetate (30 ml×3). The combined organic phases were washed with brine, dried over MgSO₄. Then the solvent was removed under reduced pressure. The crude product was separated with silica gel column chromatography (hexane/EtOAc—98/2 then 95/5) to obtain two fractions of the title product.

The first fraction was a mixture of (1R,2S,5R)-2-isopropyl-5-methylcyclohexyl (2R,3R,4S)-2-(3-bromo-4-fluorophenyl)-4-(3,4,5-trichlorophenyl)-4-(trifluoromethyl)-3,4-dihydro-2H-p yrrole-3-carboxylate and (1R,2S,5R)-2-isopropyl-5-methylcyclohexyl (2S,3S,4R)-2-(3-bromo-4-fluorophenyl)-4-(3,4,5-trichlorophenyl)-4-(trifluoromethyl)-3,4-dihydro-2H-pyrrole-3-carboxylate (380 mg, 32.3% yield).

¹H-NMR (CDCl₃) δ: 0.50-1.62 (18H, m), 3.29-3.40 (1H, m), 4.40-4.65 (1H, m), 5.63-5.80 (1H, m), 7.11-7.64 (6H, m).

The second fraction was a mixture of (1R,2S,5R)-2-isopropyl-5-methylcyclohexyl (2S,3R,4S)-2-(3-bromo-4-fluorophenyl)-4-(3,4,5-trichlorophenyl)-4-(trifluoromethyl)-3,4-dihydro-2H-p yrrole-3-carboxylate and (1R,2S,5R)-2-isopropyl-5-methylcyclohexyl (2R,3S,4R)-2-(3-bromo-4-fluorophenyl)-4-(3,4,5-trichlorophenyl)-4-(trifluoromethyl)-3,4-dihydro-2H-pyrrole-3-carboxylate (240 mg, 20.4% yield).

¹H-NMR (CDCl₃) δ: 0.17-1.56 (18H, m), 3.90-4.00 (1H, m), 4.09 (1H, d), 5.60-5.69 (1H, m) 7.03-8.15 (6H, m).

### EXAMPLE 4

### Preparation mixture of (1R,2S,5R)-2-isopropyl-5-methylcyclohexyl (3S,4R)-5-(3-bromo-4-fluorophenyl)-3-(3,4,5-trichlorophenyl)-3-(trifluoromethyl)-3,4-dihydro-2H-pyrrole-4-carboxylate

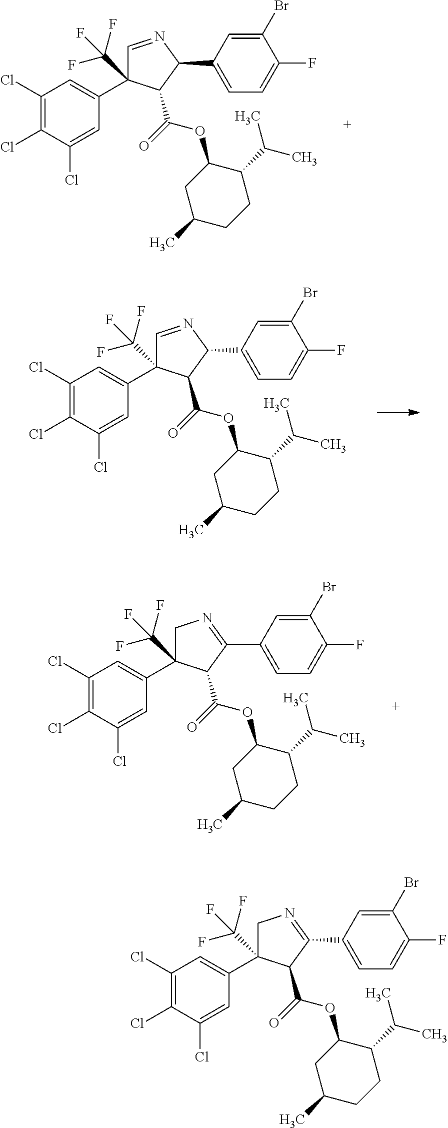

1,8-Diazabicyclo(5,4,0)undec-7-ene (68 mg, 0.45 mmol) was added to a solution of a mixture of (1R,2S,5R)-2-isopropyl-5-methylcyclohexyl (2R,3R,4S)-2-(3-bromo-4-fluorophenyl)-4-(3,4,5-trichlorophenyl)-4-(trifluoromethyl)-3,4-dihydro-2H-pyrrole-3-carboxylate and (1R,2S,5R)-2-isopropyl-5-methylcyclohexyl (2S,3S,4R)-2-(3-bromo-4-fluorophenyl)-4-(3,4,5-trichlorophenyl)-4-(trifluoromethyl)-3,4-dihydro-2H-pyrrole-3-carboxylate (150 mg, 0.22 mmol) in THF (10 ml) and the mixture was stirred over night at room temperature. After that the solvent was removed under reduced pressure, the crude product was purified with silica gel column chromatography (hexane/EtOAc—95/5) to obtain the title compound (mixture) (150 mg quantitative).

¹H-NMR (CDCl₃) δ: 0.24-1.54 (18H, m), 4.33-4.45 (1H, m), 4.60-4.91 (3H, m), 7.16-8.21 (5H, m)

### EXAMPLE 5

### Preparation of a mixture of (3S)-5-(3-bromo-4-fluorophenyl)-3-(3,4,5-trichlorophenyl)-3-(trifluoromethyl)-3,4-dihydro-2H-pyrrole

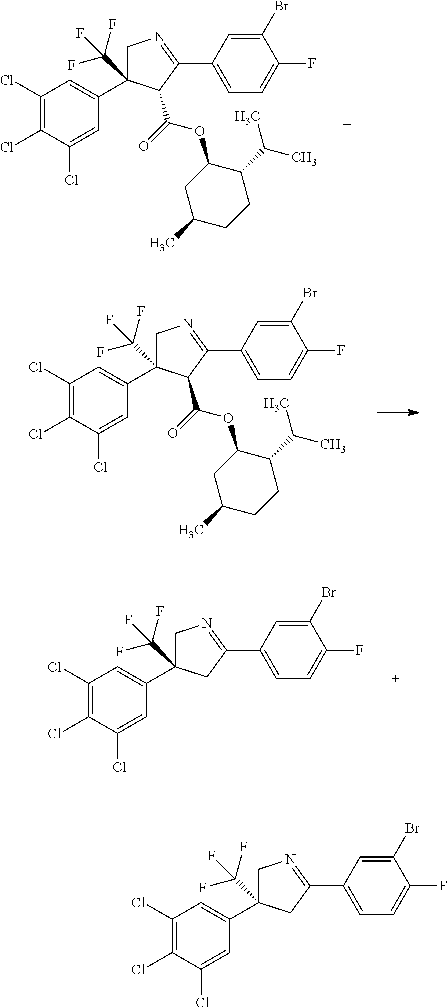

4-Toluenesulfonic acid monohydrate (130 mg, 0.67 mmol) was added in a mixture of (1R,2S,5R)-2-isopropyl-5-methylcyclohexyl(3S,4R)-5-(3-bromo-4-fluorophenyl)-3-(3,4,5-trichlorophen yl)-3-(trifluoromethyl)-3,4-dihydro-2H-pyrrole-4-carboxylate and (1R,2S,5R)-2-isopropyl-5-methylcyclohexyl(3R,4S)-5-(3-bromo-4-fluorophenyl)-3-(3,4,5-trichlorophenyl)-3-(trifluoromethyl)-3,4-dihydr o-2H-pyrrole-4-carboxylate (150 mg, 0.22 mmol) and chlorobenzene (10 ml). This mixture was refluxed for 8 hours. 4-Toluenesulfonic acid monohydrate (100 mg) was added, and then refluxed for more than 4 hours. The reaction mixture was cooled to room temperature and dissolved in, 50 ml of EtOAc, then washed with water, then brine, dried over MgSO₄. The solvent was removed under reduced pressure and the crude product was purified with silica gel chromatography (hexane/EtOAc—97/3 then 95/5) to obtain title compound (mixture) (100 mg, 91.5% yield).

¹H-NMR (CDCl₃) δ: 3.40 (1H, d), 3.75 (1H, dd), 4.25 (1H, d), 4.76 (1H, dd), 7.17-8.09 (5H, m).

### EXAMPLE 6

### Preparation of a mixture of (3S)-5-(3-bromo-4-fluorophenyl)-3-(3,4,5-trichlorophenyl)-3-(trifluoromethyl)-3,4-dihydro-2H-pyrrole

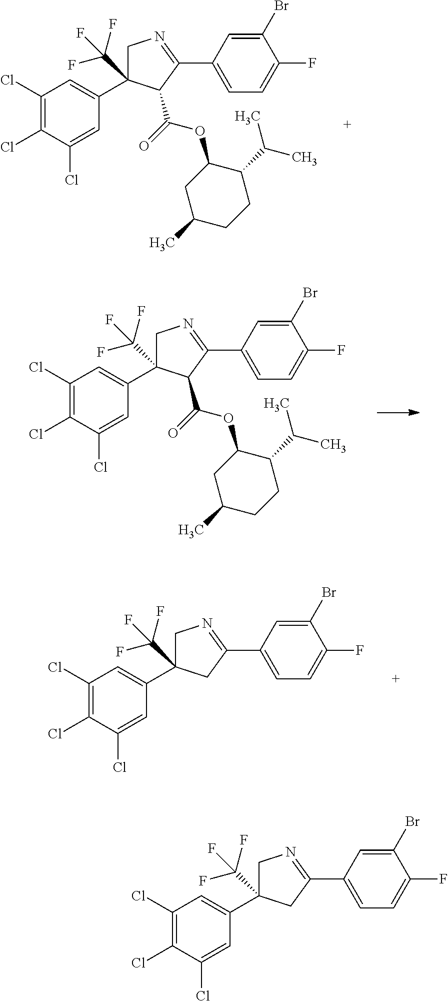

4-Toluenesulfonic acid monohydrate (234 mg, 1.22 mmol) was added in a mixture of (1R,2S,5R)-2-isopropyl-5-methylcyclohexyl(3S,4R)-5-(3-bromo-4-fluorophenyl)-3-(3,4,5-trichlorophen yl)-3-(trifluoromethyl)-3,4-dihydro-2H-pyrrole-4-carboxy late with (1R,2S,5R)-2-isopropyl-5-methylcyclohexyl (3R,4S)-5-(3-bromo-4-fluorophenyl)-3-(3,4,5-trichlorophenyl)-3-(trifluoromethyl)-3,4-dihydro-2H-pyrrole-4-carboxylate (165 mg, 0.24 mmol) and toluene (5 ml). Mixture was refluxed for 13 hours.

The reaction mixture was cooled to room temperature and dissolved in 50 ml of EtOAc, washed with water, then brine, dried over. MgSO₄. The solvent was removed under reduced pressure, the crude product was purified with silica gel chromatography (hexane/EtOAc—97/3 then 95/5) to obtain title compound (100 mg, 83.2% yield).

¹H-NMR (CDCl₃) δ: 3.40 (1H, d), 3.75 (1H, dd), 4.25 (1H, d), 4.76 (1H, dd), 7.17-8.09 (5H, m)

### EXAMPLE 7

### Preparation of (1R,2S,5R)-2-isopropyl-5-methylcyclohexyl 2-phenyl-4-(3,4,5-trichlorophenyl)-4-(trifluoromethyl)-3,4-dihydro-2H-pyrrole-3-carboxylate

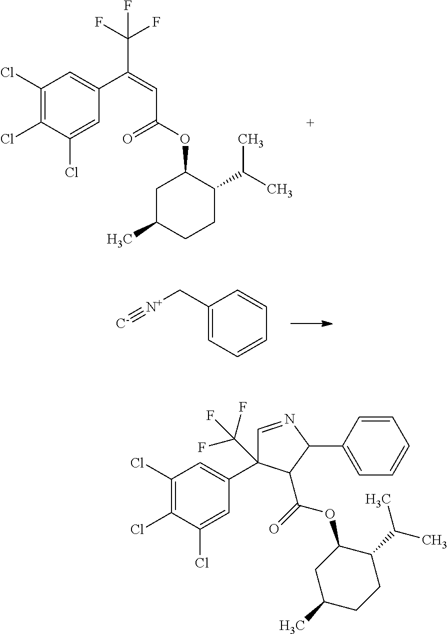

A mixture of (1R,2S,5R)-2-isopropyl-5-methylcyclohexyl (2E)-4,4,4-trifluoro-3-(3,4,5-trichlorophenyl)but-2-enoate (1.17 g, 2.55 mmol), isocyanomethylbenzene (305 mg, 2.55 mmol), copper(I)oxide (32 mg, 0.22 mmol) and toluene (20 ml) were refluxed for 6 hours. The reaction mixture was poured into water (50 ml) then extracted with ethyl acetate (30 ml×3). The combined organic phases were washed with brine, dried over MgSO₄. The solvent was removed under reduced pressure; the crude product was purified with silica gel chromatography (hexane/EtOAc—98/2 then 95/5) to obtain the title product (490 mg, 33% yield).

¹H-NMR (CDCl₃) δ: 0.43-1.63 (18H, m), 3.36-3.49 (1H, m) 4.45-5.55 (1H, m), 5.71-5.80 (1H, m), 7.21-7.64 (8H, m).

### EXAMPLE 8

### Preparation of (1R,2S,5R)-2-isopropyl-5-methylcyclohexyl 2-(3-bromo-4-fluorophenyl)-4-(3,4,5-trichlorophenyl)-4-(trifluoromethyl)-3,4-dihydro-2H-pyrrole-3-carboxylate

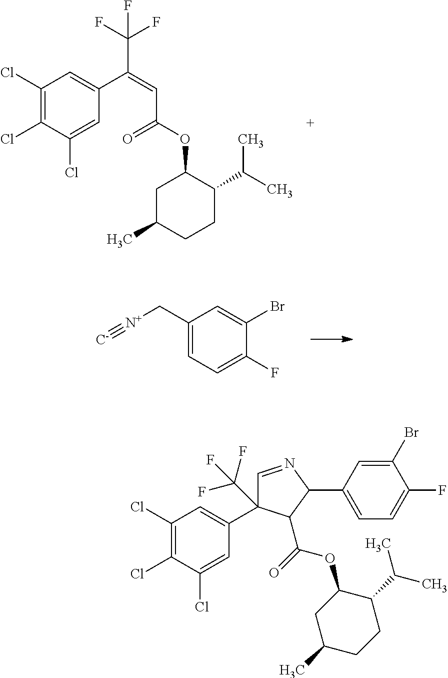

A mixture of (1R,2S,5R)-2-isopropyl-5-methylcyclohexyl (2E)-4,4,4-trifluoro-3-(3,4,5-trichlorophenyl)but-2-enoate (800 mg, 1.75 mmol), 2-bromo-1-fluoro-4-(isocyanomethyl)benzene (380 mg, 1.75 mmol), copper(I)oxide (25 mg, 0.175 mmol) and toluene (20 ml) were refluxed for 9 hours. The reaction mixture was poured into water (50 ml) then extracted with ethyl acetate (30 ml×3). The combined organic phases were washed with brine, dried over MgSO₄ The solvent was removed under reduced pressure, the crude product was purified with silica gel chromatography (hexane/EtOAc—98/2 then 95/5) to obtain the title compound (825 mg, 70.3% yield).

¹H-NMR (CDCl₃) δ: 0.50-1.62 (18H, m), 3.29-3.40 (m) & 4.09 (d) (1H), 3.90-4.00 & 4.40-4.65 (1H, m), 5.60-5.80 (1H, m), 7.11-7.64 (6H, m).

### EXAMPLE 9

### Preparation of (1R,2S,5R)-2-isopropyl-5-methylcyclohexyl-5-(3-bromo-4-fluorophenyl)-3-(3,4,5-trichlorophenyl)-3-(trifluoromethyl)-3,4-dihydro-2H-pyrrole-4-carboxylate

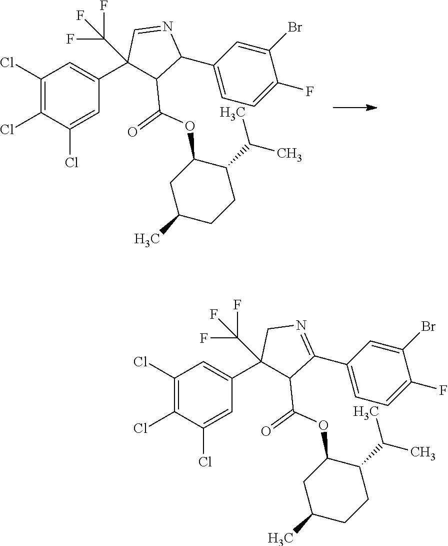

The mixture of (1R,2S,5R)-2-isopropyl-5-methylcyclohexyl-2-(3-bromo-4-fluorophenyl)-4-(3,4,5-trichlorophenyl)-4-(trifluoromethyl)-3,4-dihydro-2H-pyrrole-3-carboxylate (820 mg, 1.22 mmol), 1,8-Diazabicyclo(5,4,0)undec-7-ene (18 mg, 0.12 mmol) and pyridine (10 ml) were refluxed for 8 hours. The solvent was removed under reduced pressure; the crude product was purified with silica gel chromatography (hexane/EtOAc—95/5) to obtain the title compound (655 mg, 80% yield).

¹H-NMR (CDCl₃) δ: 0.24-1.54 (18H, m), 4.33-4.45 (1H, m), 4.60-4.91 (3H, m), 7.16-8.21 (5H, m).

### EXAMPLE 10

### Preparation of 5-(3-bromo-4-fluorophenyl)-3-(3,4,5-trichlorophenyl)-3-(trifluoromethyl)-3,4-dihydro-2H-pyrrole

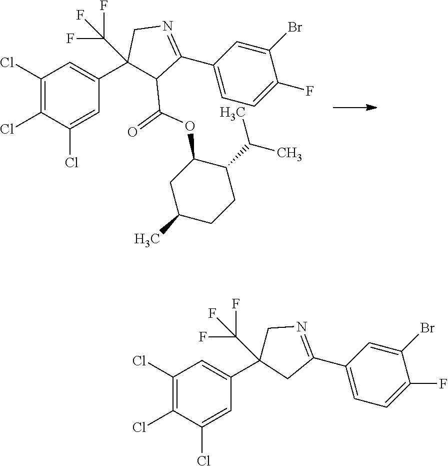

4-Toluenesulfonic acid monohydrate (890 mg, 4.69 mmol) was added in a mixture of (1R,2S,5R)-2-isopropyl-5-methylcyclohexyl 5-(3-bromo-4-fluorophenyl)-3-(3,4,5-trichlorophenyl)-3-(trifluoromethyl)-3,4-dihydro-2H-pyrrole-4-carboxylate (630 mg, 0.94 mmol) and toluene (15 ml). The mixture was refluxed for 9 hours. The reaction mixture was cooled to room temperature and dissolved in 50 ml of EtOAc, washed with water, then brine, and dried over MgSO₄. The solvent was removed under reduced pressure; the crude product was purified with silica gel chromatography (hexane/EtOAc—95/5) to obtain the title compound (420 mg, 91.5% yield).

¹H-NMR (CDCl₃) δ: 3.40 (1H, d), 3.75 (1H, dd), 4.25 (1H, d), 4.76 (1H, dd), 7.17-8.09 (5H, m)

HPLC with chiral phase (Chiracel OJ-H; Chiral Technologies): two Isomers 44.9% (+) enantiomer and 53.1 (−) enantiomer.

### EXAMPLE 11

### Preparation of (1S,2R,5S)-2-isopropyl-5-methylcyclohexyl (2E)-4,4,4-trifluoro-3-(3,4,5-tri, chlorophenyl)but-2-enoate

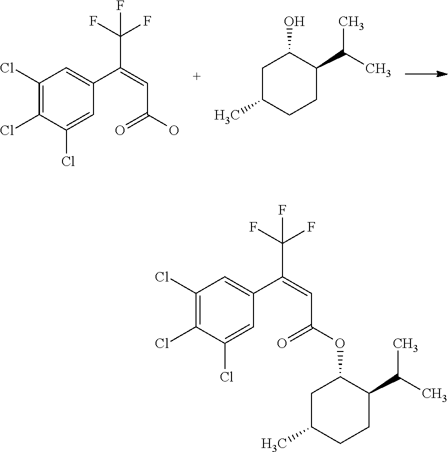

### (1) Preparation of the Acid Chloride

A mixture of (2E)-4,4,4-trifluoro-3-(3,4,5-trichlorophenyl)but-2-enoic acid (3.9 g, 10.9 mmol), dichloromethane (50 ml) and a catalytic amount of N,N-dimethylformamide was stirred at room temperature, a solution of oxalyl chloride (2.0 g, 16 mmol) in dichloromethane (20 ml) was added dropwise.

The reaction was allowed to be stirred at the same temperature for 1 hour. Solvent was removed under reduced pressure and residual solvent was removed with toluene to obtain the corresponding acid chloride, which was used for the next step without further purification.

### (2) Preparation of the Title Compound of Formula (II)

Dichloromethane (50 ml) and (1S,2R,5S)-2-isopropyl-5-methylcyclohexanol (2.06 g, 13.1 mmol) were added to the crude acid chloride then stirred in a ice-water bath. Triethylamine (1.67 g, 16.4 mmol) was added drop wise.

The reaction was allowed to be warmed up to room temperature, then stirred 1 hour.

Water (100 ml) was added, then separated; water phase was extracted with dichloromethane (50 ml×2). The combined organic phases were washed with brine then dried over MgSO₄. Solvent was removed under reduced pressure then purified with silica gel column chromatography (hexane/ethyl acetate—98/2) to obtain title compound (4.29 g, 85% yield).

¹H-NMR (CDCl₃) δ: 0.65-1.87 (18H, m), 4.59-4.68 (1H, m), 6.66 (1H, s), 7.32 (2H, s)

### EXAMPLE 12

### Preparation of (1S,2R,5S)-2-isopropyl-5-methylcyclohexyl 1-(3-bromo-4-fluorophenyl)-3-(3,4,5-trichlorophenyl)-3-(trifluoromethyl)-2,3-dihydro-1H-pyrrole-2-carboxylate

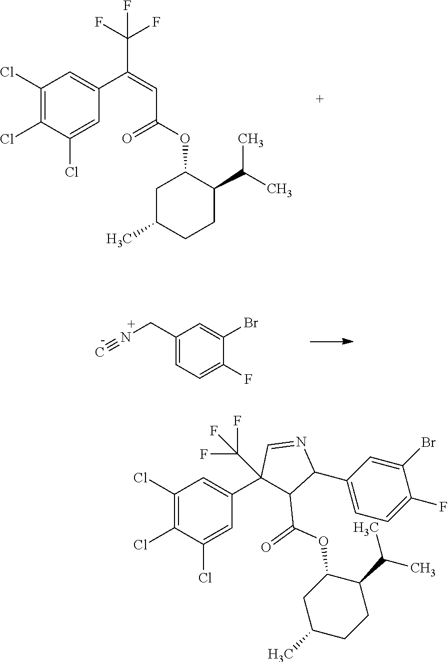

A mixture of (1S,2R,5S)-2-isopropyl-5-methylcyclohexyl (2E)-4,4,4-trifluoro-3-(3,4,5-trichlorophenyl)but-2-enoate (600 mg, 1.31 mmol), 2-bromo-1-fluoro-4-(isocyanomethyl)benzene (280 mg, 1.31 mmol) copper(I)oxide (38 mg, 0.26 mmol) and toluene (20 ml) were refluxed for 9 hours. The reaction mixture was poured into water (50 ml) then extracted with ethyl acetate (30 ml×3), the combined organic phases were washed with brine, dried over MgSO₄. Solvent was removed under reduced pressure. The crude product was then purified with silica gel column chromatography (hexane/EtOAc—98/2 then 95/5) to obtain title compound (663 mg, 75% yield).

¹H-NMR (CDCl₃) δ: 0.50-1.62 (18H, m), 3.29-3.40 (m) & 4.09 (d) (1H), 3.90-4.00 & 4.40-4.65 (1H, m), 5.60-5.80 (1H, m), 7.11-7.64 (6H, m).

### EXAMPLE 13

### Preparation of (1S,2R,5S)-2-isopropyl-5-methylcyclohecyl-5-(3-bromo-4-fluorophenyl)-3-(3,4,5-trichlorophenyl)-3-(trifluoromethyl)-3,4-dihydro-2H-pyrrole-4-carboxylate

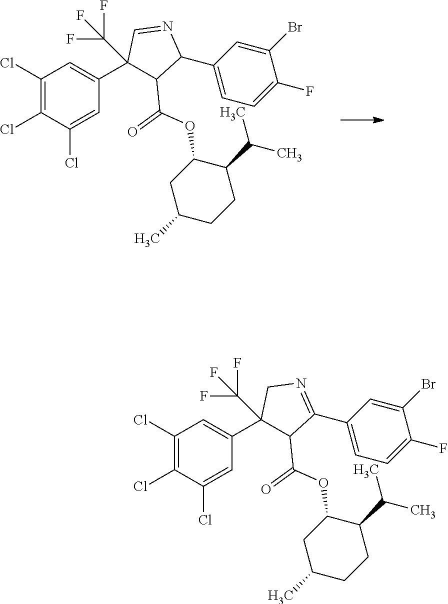

The mixture of (1S,2R,5S)-2-isopropyl-5-methylcyclohexyl-2-(3-bromo-4-fluorophenyl)-4-(3,4,5-trichlorophenyl)-4-(trifluoromethyl)-3,4-dihydro-2H-pyrrole-3-carboxylate (660 mg, 0.98 mmol), 1,8-Diazabicyclo(5,4,0)undec-7-ene (30 mg, 0.19 mmol) and pyridine (10 ml) were refluxed for 8 hours. Solvent was removed under reduced pressure. The crude product was then purified with silica gel column chromatography (hexane/EtOAc—95/5) to obtain title compound (540 mg, 81.7% yield).

¹H-NMR (CDCl₃) δ: 0.24-1.54 (18H, m), 4.33-4.45 (1H, m), 4.60-4.91 (3H, m), 7.16-8.21 (5H, m)

### EXAMPLE 14

### Preparation of 5-(3-bromo-4-fluorophenyl)-3-(3,4,5-trichlorophenyl)-3-(trifluoromethyl)-3,4-dihydro-2H-pyrrole

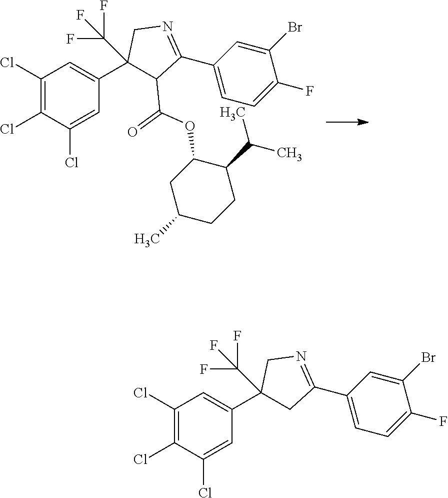

4-Toluenesulfonic acid monohydrate (766 mg, 4.03 mmol) was added to a mixture of (1S,2R,5S)-2-isopropyl-5-methylcyclohexyl 5-(3-bromo-4-fluorophenyl)-3-(3,4,5-trichlorophenyl)-3-(trifluoromethyl)-3,4-dihydro-2H-pyrrole-4-carboxylate (540 mg, 0.8 mmol) and toluene (15 ml). Mixture was refluxed for 9 hours.

Reaction mixture was cooled to room temperature and dissolved in 50 ml of EtOAc, then washed with water, then brine, and dried over MgSO₄. Solvent was removed under reduced pressure. The crude product was then purified with silica gel column chromatography (hexane/EtOAc—95/5) to obtain title compound (310 mg, 78% yield).

¹H-NMR (CDCl₃) δ: 3.40 (1H, d), 3.75 (1H, dd), 4.25 (1H, d), 4.76 (1H, dd), 7.17-8.09 (5H, m).

1. A method for the preparation of a compound of formula (I) 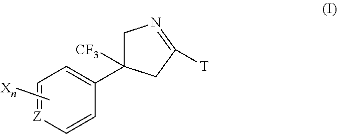
wherein T is 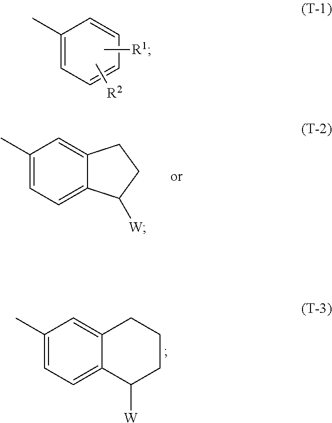
n is 1, 2, 3, 4, or 5;
Z is carbon or a nitrogen which is optionally substituted with X;
X is halogen, C₁-C₁₂-alkyl, C₁-C₁₂-haloalkyl, or C₁-C₁₂-haloalkoxy;
W is —NHCOR⁶, ═NOR⁷, ═O, or —OCOR⁸;
R¹ and R² independently of each other are hydrogen, halogen, C₁-C₁₂-alkyl, C₁-C₁₂-haloalkyl, C₁-C₁₂-alkoxy, C₁-C₁₂-alkoxycarbonyl, CH₂NHCOR³, CH(CH₃)NHCOR³, CONHR⁴, CH₂OCOR⁵, —CH(CH₃)OCOR⁵, cyano, nitro, triazolyl, or tetrazolyl;
R³, R⁵, R⁶, R⁷, and R⁸, independently of each other, are C₁-C₁₂-alkyl, C₂-C₁₂-alkenyl, arylalkyl, C₁-C₁₂-haloalkyl, C₃-C₇-cycloalkyl, C₃-C₇-cycloalkyl(C₁-C₁₂)-alkyl, C₁-C₁₂-alkyl(C₁-C₁₂)-thioalkyl, C₁-C₁₂-alkylsulfinyl(C₁-C₁₂)-alkyl, C₁-C₁₂-alkylsulfonyl(C₁-C₁₂)-alkyl, optionally substituted phenyl, or optionally substituted 3- to 7-membered heterocycle containing at least one nitrogen, oxygen, or sulfur; and
R⁴ is C₁-C₁₂-alkyl, C₂-C₁₂-alkenyl, arylalkyl, C₁-C₁₂-haloalkyl, C₃-C₇-cycloalkyl, C₃-C₇-cycloalkyl(C₁-C₁₂)-alkyl, or a 3- to 7-membered heterocycle containing at least one nitrogen, oxygen, or sulfur;
which method comprises:
(i) reacting a chiral compound having the general formula (II) 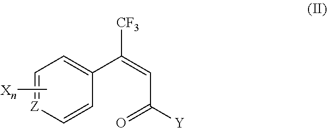
wherein
X, n, and Z have the meanings as defined above, and
Y is —OR⁹ or —NR¹⁰R¹¹; in which
R⁹ and R¹¹ independently of each other are optically active C₃-C₁₂-cycloalkyl or optically active —CR¹²R¹³R¹⁴;
R¹⁰ is hydrogen or C₁-C₁₂-alkyl; and
R¹², R¹³, and R¹⁴ independently of each other are hydrogen, C₁-C₁₂-alkyl, C₁-C₁₂-alkoxycarbonyl, C₃-C₇-cycloalkyl, or optionally substituted phenyl;
with a compound of the general formula (III) 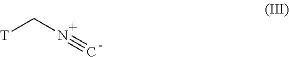
wherein T has the meaning as defined above
in the presence of a metal catalyst, and optionally in the presence of a base, to obtain a compound having the general formula (IV) 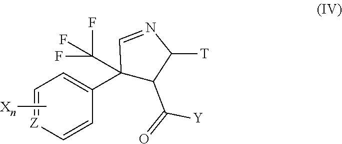
wherein X, n, Z, Y, and T have the meanings as defined above;
(ii) isomerizing the compound of formula (IV) to obtain a compound having the general formula (V) 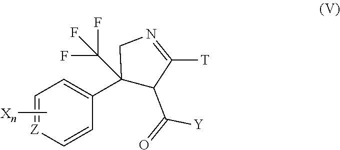
wherein X, n, Z, Y, and T have the meanings as defined above; and
(iii) reacting the compound of formula (V) under acidic conditions, to obtain the compound of formula (I).

2. The method according to claim 1, wherein the metal catalyst used in (i) is selected from the group consisting of copper(I)oxide, copper(I)cyanide, copper(I)chloride, copper(I)bromide, copper(I)iodide, copper(I)acetate, copper(II)cyanide, copper(II)chloride, copper(II)bromide, copper(II)iodide, copper(II)oxide, copper(II)acetate, and copper(II)acetylacetonate.

3. The method according to claim 1, wherein the isomerisation reaction of (ii) is conducted in the presence of an organic or inorganic base, and wherein the molar ratio of compound (IV) and the base is in the range from 0.05 to 10.

4. A compound of formula (II) 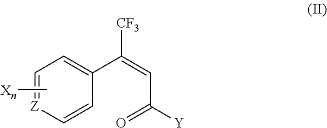
wherein
X is halogen, C₁-C₁₂-alkyl, C₁-C₁₂-haloalkyl, or C₁-C₁₂-haloalkoxy;
n is 1, 2, 3, 4, or 5;
Z is carbon or a nitrogen which is optionally substituted with X;
Y is —OR⁹ or —NR¹⁰R¹¹; in which
R⁹ and R¹¹, independently of each other, are optically active C₃-C₁₂-cycloalkyl or optically active —CR¹²R¹³R¹⁴;
R¹⁰ is hydrogen or C₁-C₁₂-alkyl; and
R¹², R¹³, and R¹⁴ independently from each other are hydrogen, C₁-C₁₂-alkyl, C₁-C₁₂-alkoxycarbonyl, C₃-C₇-cycloalkyl, or optionally substituted phenyl.

5. The compound according to claim 4, wherein
X is C₁-C₆-alkyl, F, Cl, Br, I, trifluoromethyl, difluoromethoxy, or methoxy;
n is 1, 2, 3, 4, or 5;
Z is carbon or a nitrogen which is optionally substituted with X;
Y is —O-menthyl, —O-α-methylbenzyl, —N(R¹⁰)(menthyl), or —N(R¹⁰)(α-methylbenzyl); and
R¹⁰ is hydrogen or C₁-C₁₂-alkyl.

6. A compound of general formula (IV) 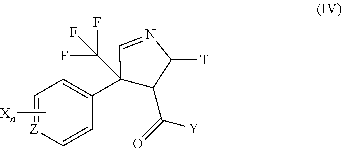
wherein T is 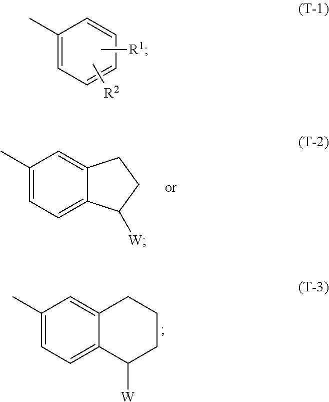
n is 1, 2, 3, 4, or 5;
Z is carbon or a nitrogen which is optionally substituted with X;
X is halogen, C₁-C₁₂-alkyl, C₁-C₁₂-haloalkyl, or C₁-C₁₂-haloalkoxy;
W is —NHCOR⁶, ═NOR⁷, ═O, or —OCOR⁸;
R¹ and R² independently of each other are hydrogen, halogen, C₁-C₁₂-alkyl, C₁-C₁₂-haloalkyl, C₁-C₁₂-alkoxy, C₁-C₁₂-alkoxycarbonyl, CH₂NHCOR³, CH(CH₃)NHCOR³, CONHR⁴, CH₂OCOR⁵, —CH(CH₃)OCOR⁵, cyano, nitro, triazolyl, or tetrazolyl;
R³, R⁵, R⁶, R⁷, and R⁸, independently of each other, are C₁-C₁₂-alkyl, C₂-C₁₂-alkenyl, arylalkyl, C₁-C₁₂-haloalkyl, C₃-C₇-cycloalkyl, C₃-C₇-cycloalkyl(C₁-C₁₂)-alkyl, C₁-C₁₂-alkyl(C₁-C₁₂)-thioalkyl, C₁-C₁₂-alkylsulfinyl(C₁-C₁₂)-alkyl, C₁-C₁₂-allylsulfonyl(C₁-C₁₂)-alkyl, optionally substituted phenyl, or optionally substituted 3- to 7-membered heterocycle containing at least one nitrogen, oxygen, or sulfur;
R⁴ is C₁-C₁₂-alkyl, C₂-C₁₂-alkenyl, arylalkyl, C₁-C₁₂-haloalkyl, C₃-C₇-cycloalkyl, C₃-C₇-cycloalkyl(C₁-C₁₂)-alkyl, or a 3- to 7-membered heterocycle containing at least one nitrogen, oxygen, or sulfur;
Y is —OR⁹ or —NR¹⁰R¹¹, in which
R⁹ and R¹¹ independently of each other are optically active C₃-C₁₂-cycloalkyl or optically active —CR¹²R¹³R¹⁴;
R¹⁰ is hydrogen or C₁-C₁₂-alkyl; and
R¹², R¹³, and R¹⁴ independently of each other are hydrogen, C₁-C₁₂-alkyl, C₁-C₁₂-alkoxycarbonyl, C₃-C₇-cycloalkyl, or optionally substituted phenyl.

7. The method of claim 1, wherein the compound of formula (I) is selected from the group consisting of
(1R,2S,5R)-2-isopropyl-5-methylcyclohexyl-5-(3-bromo-4-fluorophenyl)-3-(3,4,5-trichlorophenyl)-3-(trifluoromethyl)-3,4-dihydro-2H-pyrrole-4-carboxylate; and
(1R,2S,5S)-2-isopropyl-5-methylcyclohexyl-5-(3-bromo-4-fluorophenyl)-3-(3,4,5-trichlorophenyl)-3-(trifluoromethyl)-3,4-dihydro-2H-pyrrole-4-carboxylate.
